(19) United States
(12) Patent Application Publication
(10) Pub. No.: US 2012/0282677 A1
(43) Pub. Date: Nov. 8, 2012

(54) PHOTOBIOREACTOR COMPRISING ROTATIONALLY OSCILLATING LIGHT SOURCES

(75) Inventors: Helmut BROD, Koln (DE); Bjoern FRAHM, Lemgo (DE)

(73) Assignee: BAYER INTELLECTUAL PROPERTY GMBH, Monheim (DE)

(21) Appl. No.: 13/462,531

(22) Filed: May 2, 2012

(30) Foreign Application Priority Data
May 3, 2011 (DE) 102011075110.6

(51) Int. Cl. C12M 1/42; C12N 1/12

(52) U.S. Cl. 435/257.1; 435/292.1

(57) ABSTRACT

The invention relates to a photobioreactor having illumination by light sources (light guides, LEDs) moved in rotational oscillation, and optionally membrane surfaces for gas transport moved in combination in rotational oscillation.

Advantages are, inter alia, light input which is spatially homogeneous on average over time and can be adapted by means of intensity and oscillation to the culture and its density, as well as low-shear power input and optionally low-shear bubble free gasification and degassing.

### CROSS REFERENCE TO RELATED APPLICATION

This application claims the priority to German Application No. 102011075110.6, filed May 3, 2011, the content of which is incorporated herein by reference in its entirety.

### BACKGROUND

1. Field of the Invention

The present invention relates to a photobioreactor which comprises light sources mobile in rotational oscillation inside the reactor for spatially homogeneous light input.

2. Description of Related Art

The invention may be used for the cultivation of phototrophic organisms, which are irradiated in an optimized fashion by the light sources mobile in rotational oscillation inside the reactor.

This optimized light input may optionally be combined with optimized gasification and degassing for shear-sensitive reactor content substances such as shear-sensitive phototrophic organisms.

One important field of application of phototrophic organisms is the cultivation of algae. Besides the traditional cultivation of algae in flat containers exposed to sunlight, the cultivation of algae in bioreactors is increasingly being described. One reason for this development is the use of algae to produce very high-value products. This, however, entails much more complex requirements for the cultivation conditions, or the process management. It can only be carried out in specially designed photobioreactors. The economic viability of the higher procurement and production costs resulting from this, in comparison with the cheaper, traditional cultivation containers, depends on the achievable high price level of the products.

Such high-value products are, for example, colourants in the cosmetics industry or polyunsaturated fatty acids such as natural omega-3 fatty acids or eicosapentaenoic acid (EPA) in the food supplement industry. Proteins for therapeutic and diagnostic pharmaceuticals are under development in the pharmaceutical industry. Another field is green energy production (for example production of hydrogen by the algae). The algal biomass, which is formed anyway in the production of products from algae, can furthermore be used for the production of biogas, which makes this technology particularly environmentally friendly. Another example of the combination of algal cultivation and utilization of the algal biomass for biogas production is off-gas purification, in which the carbon dioxide contained and the carbon are metabolized by the algae. The environmental technology process of off-gas purification can therefore be symbiotically linked with the energy technology aspect of obtaining biogas from algal biomass which has been produced.

Photobioreactors of various designs are described in the prior art.

Havel et al. describe a bioreactor comprising up to 16 bubble columns operated in parallel, which are equipped with an artificial light source directly over the bubble columns so that a sufficient light supply is ensured over a homogeneous light spectrum. Around each bubble column, reflective cylindrical tubes convey the light from above to the cylindrical glass outer walls of each bubble column, in order to reduce the light scattering and improve the irradiation. A combination of Osram Fluora L77 (Osram, Munich, Germany) and Sun-Glo (Hagen, Holm, Germany) fluorescent tubes are used as light source to generate artificial sunlight [J. Havel, E. Franco-Lara, D. Weuster-Botz: “A parallel bubble column system for the cultivation of phototrophic microorganisms” Biotechnology Letters (2008) 30:1197-1200]. Inside the bubble column, the flow dynamics control the light exposure of the floating photosynthetic cells, with the organisms moving quasi-chaotically in the bubble column. At optimal performance, the bubble columns must furthermore be operated with the highest possible gasification rate, which is dictated by the shear tolerance of the algae. At the same time, the gasification rate should not be so high that gas stagnation can take place, which would prevent the transmission of light through the bubble column [J. C. Merchuk, F. Garcia-Camacho, E. Molina-Grima “Photobioreactor Design and Fluid Dynamics” Chem. Biochemical Engineering Quarterly (2007) 21 (4):345-355]. Such photobioreactors are nevertheless those most widely used, because they are simple to construct and to keep in operation. However, scale-up and optimal control of this type of photoreactor are difficult so that their productivity is usually low. The reason for this is also based on the fact that the light input in these photobioreactors takes place through the outer surface of the reactor. With increasing scale, however, the surface area to volume ratio becomes smaller and the light input per unit volume therefore becomes lower, so that the product yield is limited.

A tube photobioreactor consists of transparent tube material arranged straight or in coils, the arrangement of which is intended to achieve the maximum sun/light radiation reception. The phototrophic cultivation material is conveyed inside the tubes. For this purpose, airlift circulators are particularly often used [J. C. Merchuk, F. Garcia-Camacho, E. Molina-Grima “Photobioreactor Design and Fluid Dynamics” Chem. Biochemical Engineering Quarterly (2007) 21 (4):345-355]. Merchuk et al. furthermore give an overview of the prior art of cultivating photosynthetic cells in bioreactors. Besides the aforementioned bubble column reactors and tube reactors, thin-film bioreactors and airlift reactors are also described. In contrast to bubble columns, airlift reactors allow controlled flow through channels. In the structure consisting of concentric tubes, the light source lies on the outer walls and the inner walls define the dark region [J. C. Merchuk, F. Garcia-Camacho, E. Molina-Grima “Photobioreactor Design and Fluid Dynamics” Chem. Biochemical Engineering Quarterly (2007) 21 (4):345-355].

Rastre et al. describe a method for the rational design of large-scale reactors by combining model bioreactors and pilot bioreactors. Closed tube and plate bioreactors are studied [R. R. Sastre, Z. Csögör, I. Perner-Nochta, P. Fleck-Schneider, C. Posten, Journal of Biotechnology (2007) 132:127-133].

US 20100144019 describes a photobioreactor for the cultivation of microalgae, the container of which contains a multiplicity of light sources of different length, particularly photodiodes in combination with a light guide, a mechanical stirring means for generating flows and a pneumatic mixing system, which generates bubbles in order to suspend the microalgae, with the light sources being immersed into the container. Flexible light sources are not disclosed.

WO 2009069967 describes a photobioreactor for the cultivation of microalgae, consisting of a multiplicity of light source surfaces, for example a flexible LED sheet, in a container. The light source surfaces have the shape of a flat plate or a cylinder and are installed at regular intervals inside the reactor container so as to partition it. As in a tube reactor, the culture is conveyed into the reactor from an input to an output along the light source surfaces, the latter being placed in the container so that the path between the input and output is as long as possible.

A photobioreactor for the cultivation of algae is described in US 20100028977 A1, in which light is introduced into the culture liquid via rods. The described arrangement of the rods does not, however, allow homogeneous light input into the photobioreactor. Another disadvantage is that although rotation of the arranged rods is described, this does not involve generating a relative velocity between the light source and the culture liquid comprising the phototrophic organisms. Such a relative velocity leads to corresponding mixing, with the result that different phototrophic organisms are constantly transported into the vicinity of the light source. A relative velocity also has the effect that all the phototrophic organisms receive the same light dose on average over time, so that there are no organisms which receive a lower light dose on average over time owing to a greater distance from the light source. Patent US 20100028977 A1 furthermore lacks a gasification and degassing concept for the described photobioreactor. This is another disadvantage since, besides the light input, the supply of carbon dioxide and the discharge of oxygen is also important for algae.

US 20100028977 A1 thus has the disadvantages of light input which is not spatially homogeneous, no relative velocity between the light source and the culture liquid, and a lack of gas transfer concept.

In all the photoreactors mentioned above, one alternative is that the light source is located outside the cultivation container and does not come in contact with the cultivated material. Correspondingly, high-energy light sources are required in order to generate the necessary radiation and a maximal penetration depth of the light radiation. This direct irradiation entails the risk of photoinhibition for particularly sensitive organisms. Alternatively, as another possibility, the light is guided from the light source into the photobioreactor by light guides, or both possibilities are combined.

Despite enormous interest in recent years, an economically satisfactory solution is not yet available for the cultivation of phototrophic cells, particularly algae and cyanobacteria, in bioreactors.

A method for the bubble-free gasification of liquids, in particular cell cultures, is described in WO 2007098850 and WO 2010034428, with gas exchange via one or more membrane surfaces immersed flexibly in the medium to be gasified and/or degassed, for example tubes, cylinders or modules, wherein the membrane surface executes an arbitrary rotationally oscillating movement in the medium. The rotationally oscillating movement is in this case simple to achieve by corresponding operation of the drive motor. In design technology and mechanical terms, this entails no additional requirements. By controlled modification of the movement, the movement can be optimized so that the flow onto the membrane surface is optimal. Since the material transport coefficient depends on the flow onto the membrane surface, an optimal movement is one in which the membrane surface respectively has a maximal relative velocity with respect to the liquid. Another advantage of the rotationally oscillating movement of the membrane surface is the fact that a separate stirring or mixing member for generating a flow onto the membrane surface is obviated. Furthermore, the rotationally oscillating movement means that flow baffles in the container are not necessary. In conventional stirring members, flow baffles are conventionally used in order to prevent the liquid from moving with the stirrer and to provide sufficient turbulence and power input. This combination of providing a membrane surface and generating a flow onto the membrane surface in WO 2007098850 and WO 2010034428 avoids zones with locally high power input and locally high shear stress. In the described bioreactor, the power input takes place in a spatially uniform pattern and is used directly for the flow onto the membrane surface. With expedient liquid movement at all positions on the membrane surface, a high and defined material transport takes place owing to the movement of the membrane surfaces relative to the liquid. Overall, a better relationship between material transport and the mechanical power input required for generating the material transport, and the inevitable shear stress in the bioreactor, is achieved compared with conventional methods and devices. Use of the bioreactor for the cultivation of phototrophic cells is not described, and a light source is not provided in the described device.

In contrast to the cultivation of bacteria, yeasts or mammalian cells in bioreactors, the cultivation of phototrophic organisms such as algae in photobioreactors requires the input of light energy in addition to the gas supply and discharge. Owing to the short penetration depth of the light radiation, maximally homogeneous light input is required with there being a far as possible no regions of the bioreactor in which the light source is so far away that the productivity is thereby compromised. The exponential attenuation of the irradiation strength produces three zones with different growth conditions:

In the first zone, which extends from the light source to the point at which the light energy covers the energy demand of the alga for maximal growth rate, the growth rate then depends primarily on the cell type and cultivation medium. Under certain circumstances, photoinhibition in this zone may prevent maximal growth in the vicinity of the light source.
The second zone ends at the point at which the light energy reaching the cells is just equal to the energy demand for subsistence metabolism. In this zone, the light is the limiting factor and the photosynthetic growth rate is proportional to the incoming light intensity.
The third zone is the underilluminated region, in which the growth is negative and fouling takes place.

The aforementioned zone system does not take into account the flow dynamics inside the bioreactor.

For the cultivation of phototrophic organisms such as algae in photobioreactors, the object therefore arises not only to provide a high gas supply and gas discharge with sufficient mixing and avoidance of fouling and aggregation, as is customary in bioreactors, but also at the same time to achieve sufficient light input which is as homogeneous as possible.

In the photobioreactor design, besides the additional demanding aspect of the light input, depending on the phototrophic organism greater attention must furthermore be paid to the shear sensitivity.

Shear stress also occurs inter alia because of bubble gasification, so that bubble-free gasification is advantageous, particularly for phototrophic organisms which in any case prefer bubble-free gasification. For shear-sensitive and/or bubble-sensitive organisms, the object described above therefore becomes more difficult. In the case of bubble-free gasification, in particular attention must be paid to the avoidance of fouling and aggregation. Homogeneous light input with gas supply and discharge, optionally in bubble-free fashion, sufficient mixing and avoidance of fouling and aggregation together with low shear stress must thus be provided, low shear stress often also entailing low power input and therefore low gas supply and discharge as well as mixing.

Furthermore, a photobioreactor is required which can be constructed, operated and kept in operation in an economically satisfactory way, and can be adapted as flexibly as possible to the requirements of the cultivated organisms.

The demands on photobioreactors are sufficiently satisfied by the previously existing systems only for individual or several criteria, but not for all criteria.

### SUMMARY

Surprisingly, the aforementioned object has been achieved by a photobioreactor characterized by light sources arbitrarily mobile in rotational oscillation distributed in a bioreactor liquid volume, and by a method of using the photobioreactor according to the invention.

### BRIEF DESCRIPTION OF THE DRAWINGS

FIGS. 1-11 represent various embodiments of the present invention.

### DETAILED DESCRIPTION OF A PREFERRED EMBODIMENT

In this context, the term light source is not necessarily intended to mean that the light source generates the light; rather, the term light source is also used in the sense of light output. For example, the light may be generated outside the photobioreactor and conveyed by glass fibre cable into the bioreactor, where it emerges from light guides. The latter function as a light source for the photobioreactor.

The invention is suitable not only for phototrophic organisms such as algae and cyanobacteria, but also for other corresponding applications with light input and optionally gasification.

The light input (preferably in liquids) is used here to provide light for photosynthesis. By applying the light source (for example in a star shape on a rotor), in conjunction with the movement in the liquid, the problem of the short light penetration depth is resolved.

Usually, the light sources are fixed on one or more carriers. Suitable carriers are membrane surfaces such as tubes, cylinders or other modules made of preferably bioprocess-compatible steel or plastic.

Preferably, the light sources are spatially distributed approximately homogeneously. Preferably, these light sources are distributed statistically throughout the volume of the photobioreactor.

In a preferred embodiment, the light sources are distributed spatially homogeneously in the photobioreactor in the form of flexible light guides or light-emitting diodes (LEDs), for example by arrangement on a rotor branching in a star shape (see example in FIG. 11). For the case of a container fill which does not absorb light, this makes the illumination in the container equal everywhere. An advantageous mutual arrangement of the light sources in relation to the reactor walls and reactor fitments may also be calculated mathematically in order to achieve ideal lighting. The rotor carries rotor arms in a star shape for example on its upper and lower ends, i.e. for example under the liquid surface and over the bottom of the bioreactor. The flexible light guides are then for example wound vertically onto the rotor arms, for example from the upper rotor arm to the lower rotor arm, back to the upper rotor arm and so on. Fluting of the rotor arms on the bearing region can assist secure support of the light guides. Thin light guides are preferably to be used in order to maximize their surface area to volume ratio. Owing to the discontinuous movement of the rotor shaft, flow onto the light sources takes place tangentially.

The described arrangement of light sources or light guides, LEDs in the photoreactor is ideal for scale-up or scale-down of the photobioreactor, since the ratio of the light source surface area or light guide surface area to the photobioreactor volume can be kept constant.

Owing to the immediate proximity of the light source to the cells, comparatively low-energy, i.e. also energy-saving light sources are suitable.

Examples of light guides are light waveguides, glass fibres, polymeric optical fibres or other light-guiding components made of plastic, as well as fibre-optic components.

Besides this, light-emitting diodes (LEDs) may be used as light sources in the reactor. The properties of the light generated can be varied by expedient selection of the semiconductor materials and the doping. Above all, the spectral range and the efficiency can thus be influenced:

aluminium gallium arsenide (AlGaAs)—red (665 nm) and infrared up to 1000 nm wavelength,
gallium arsenide phosphide (GaAsP) and aluminium indium gallium phosphide (AlInGaP)—red, orange and yellow,
gallium phosphide (GaP)—green,
silicon carbide (SiC)—first commercial blue LED; low efficiency,
zinc selenide (ZnSe)—blue emitter, but which never reached commercial fruition,
indium gallium nitride (InGaN)/gallium nitride (GaN)—ultraviolet, violet, blue and green, white LEDs are usually blue LEDs with a fluorescent layer placed in front of them, which acts as a wavelength converter.

The present invention likewise relates to the use of light guides and/or light-emitting diodes for promoting the growth of phototrophic organisms in a photobioreactor according to the invention.

LEDs having a resultant irradiance of preferably about 5-120 μmol/m2·s, particularly preferably about 5-30 μmol/m2·s, are used in the rotationally oscillating photobioreactors.

The light sources are preferably controlled by means of a light control unit. In particular, LEDs can be switched and modulated very rapidly by means of the operating current. In this way, the light input (light energy, light intensity) can be adapted for example to the culture density. Pulsation of the light source is furthermore readily achievable.

Owing to the rotationally oscillating movement, the light sources supply all the phototrophic organisms, such as algal cells, with light as uniformly as possible on average over time. The oscillatory rotational movement generates a relative velocity between the light source and the reactor content, and has the effect that each part of the reactor content receives the same light dose on average over time. The fact that, for example with a star-shaped arrangement of the light sources, the relative velocity between the light source and the reactor content decreases with a decreasing distance from the oscillation axis is not important, since the reactor content is mixed thoroughly and a particle in the reactor will thus occupy different distances from the oscillation axis in the course of time.

The varying light intensity associated with the oscillation, which is experienced by an individual phototrophic organism cell such as an algal cell, is not critical since the irradiation of algae is in any case usually carried out in a pulsed fashion (for example with a frequency of one hertz). What is important is merely that, in this invention, all algal cells are exposed to the same sufficient amount of light on average. The technical requirement of being able to cultivate phototrophic organisms such as algae in reactors despite the short penetration depth of the light radiation is thereby accommodated. Stirring and mixing members for flow onto the light source surface are obviated, so that zones with locally high power input and locally high shear stress are avoided. This accommodates the shear sensitivity of certain phototrophic organisms (for example certain types of alga).

The oscillation may furthermore be adapted, for example, to the culture density. A higher culture density, for example of algae, leads to a shorter penetration depth of the light. This may be compensated for by amplifying the oscillation for a higher culture density, optionally in combination with an increase in the light energy of the light source. This will prevent the illumination per algal cell from decreasing with an increasing culture density.

The photobioreactor according to the invention is comparatively simple and economical to provide and can be adapted in a relatively uncomplicated way to the requirements of the organisms being cultivated.

Typically, the photobioreactor according to the invention is cylindrical with a preferred size of from 1 l to 1000 l in terms of fill volume and with standard height to diameter ratios, so that retrofitting of existing bioreactors is possible.

If bubble-free gasification for the transport of e.g. O2 or CO2 is required, then the photobioreactor according to the invention will comprise gas transport means mobile in rotational oscillation, selected from the group containing spargers, microspargers or membrane surfaces, in particular membrane tubes. The gasification membrane surface may be installed on the so-called rotor in addition to the light source carriers, for example in the form of additional rotor arms in a similar way as in the preferred embodiment explained above. As an alternative, gasification membrane surfaces, for example gasification tubes, may be equipped with one or more light sources.

The gasification of liquids is used to introduce and desorb gases. Further aims in biotechnology are, by corresponding membrane gasification, to achieve high material transport coefficients together with a low power input or together with a low shear load. The gas exchange takes place via one or more arbitrarily configured immersed membrane surfaces, the membrane surface executing an arbitrary rotationally oscillating movement in the liquid. The membrane gasification, which can be carried out bubble-free, accommodates the shear sensitivity and the demand for bubble-free gasification of certain phototrophic organisms, for example certain types of alga. Optionally, the membrane surface may also be selected so that it only assists the gas supply and discharge. The membrane surface may, for example, be formed from one or more membrane tubes.

As an alternative or in addition to the gasification membrane surface, one or more spargers or microspargers may also be applied on the arbitrarily rotationally oscillating light source, preferably at the lower end so that the bubble ascent path is longer. Furthermore, the bubbles or microbubbles for the gas transfer ascend along the light source, which additionally leads to corresponding mixing of the regions and therefore to a light input which is more uniform on average over time, since different organisms are constantly transported into the vicinity of the light source by the mixing. In conjunction with the arbitrarily rotationally oscillating movement, all regions of the bioreactor are then supplied with (micro)bubbles.

This method and this device have the further advantage that agglomeration and deposition of substances on the inner regions of the corresponding cultivation vessel/photobioreactor are avoided or greatly reduced. Such phenomena are generally disadvantageous since the function of elements in the bioreactor, for example gas transfer membranes or probes, is sometimes greatly restricted or even negated [WO 2010034428]. In the cultivation of phototrophic organisms such as algae in the photobioreactor according to the invention, deposition of substances on the light sources is furthermore avoided or greatly reduced.

Furthermore, according to the cultivation requirements of the respective algae or phototrophic organisms, not only the ratio of the light source area and the membrane area to the bioreactor volume, but also the ratio of the light source area to the membrane area, can be varied straightforwardly. In a preferred embodiment, for example, membrane tubes and light guides may be wound on rotor arms (for example alternately), in which case the aforementioned ratios may be varied by the number of rotor arms and the number of membrane tubes in relation to the number of light guides. The membrane tubes and light guides are preferably supplied by a gas supply and light source arranged outside the vessel.

In another embodiment of the invention, the photobioreactor comprises control elements by which the excursion of the membrane surface in one rotation direction can be limited, in which case the control elements may comprise light guides or light-emitting diodes.

Preferably, the light source, the gas transport means or both are furthermore connected by means of a common frame to one or more probes, which make it possible to monitor the processes inside the reactor.

The present invention furthermore relates to a method of using the photobioreactor according to the invention, characterized in that the light sources execute an arbitrary discontinuous movement, preferably an arbitrary movement with movement reversal, particularly preferably a rotationally oscillating movement.

It is furthermore preferable for the gas transport means to execute an arbitrary discontinuous movement, a movement with movement reversal or a rotationally oscillating movement. In a particular embodiment of the method, the light sources, the gas transport means or both move in a periodic sequence of acceleration and deceleration between two movement turning points.

The method according to the invention is particularly suitable for the cultivation of algae or phototrophic organisms.

The present invention furthermore relates to the use of light guides, light-emitting diodes or both to promote the growth of phototrophic organisms in a photobioreactor.

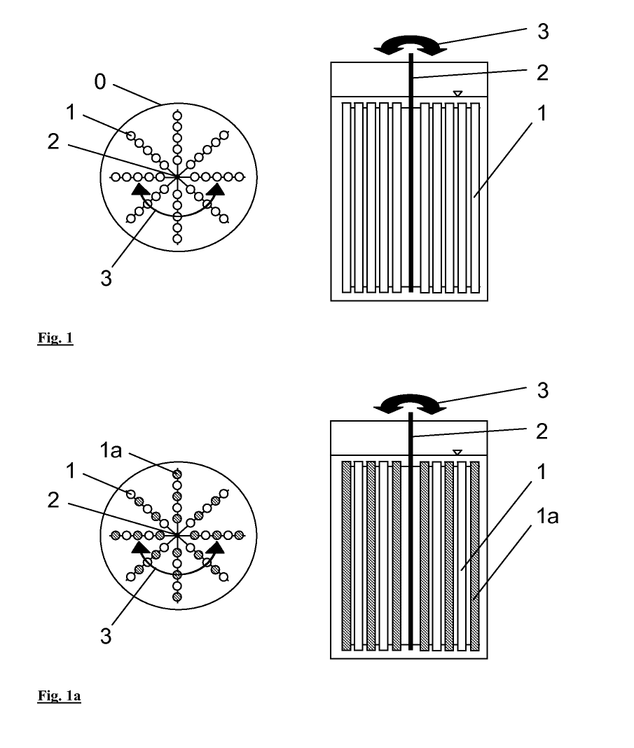

FIGS. 1 to 11 show possible embodiments of the photobioreactor according to the invention, without being limited thereto.

FIG. 1: Schematic representation of a rotationally oscillating movement for light input in a container. Light guides (1) wound on a rotor in this case form the light input surface. It rotates with the rotor shaft (2) in both rotation directions (3).

FIG. 1a: Schematic representation of a rotationally oscillating movement for light input in a container combined with gas transfer apparatus. In addition to the light guides, membrane tubes (1a) for the gas transfer are wound on the rotor (a light guide and membrane tube always alternate and alternate with an offset on each neighbouring rotor arm).

FIG. 1b: Schematic representation of a rotationally oscillating movement for light input in a container combined with gas transfer apparatus. In addition to the light guides, membrane tubes (1a) for the gas transfer are wound on the rotor (light guide on every second rotor arm and membrane tube on all the other rotor arms).

FIG. 1c: Schematic representation of a rotationally oscillating movement for light input in a container combined with gas transfer apparatus. In addition to the light guides, a sparger (1b) (for example a microsparger) is in this case arranged below the light guide surfaces.

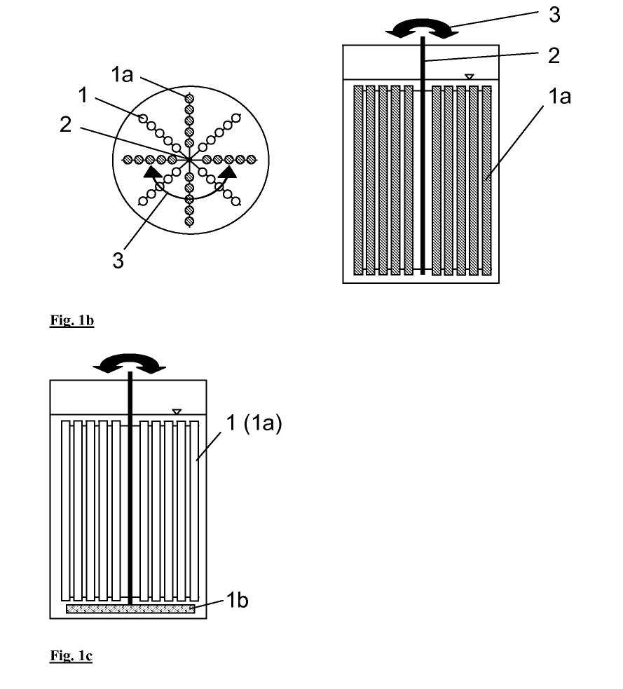

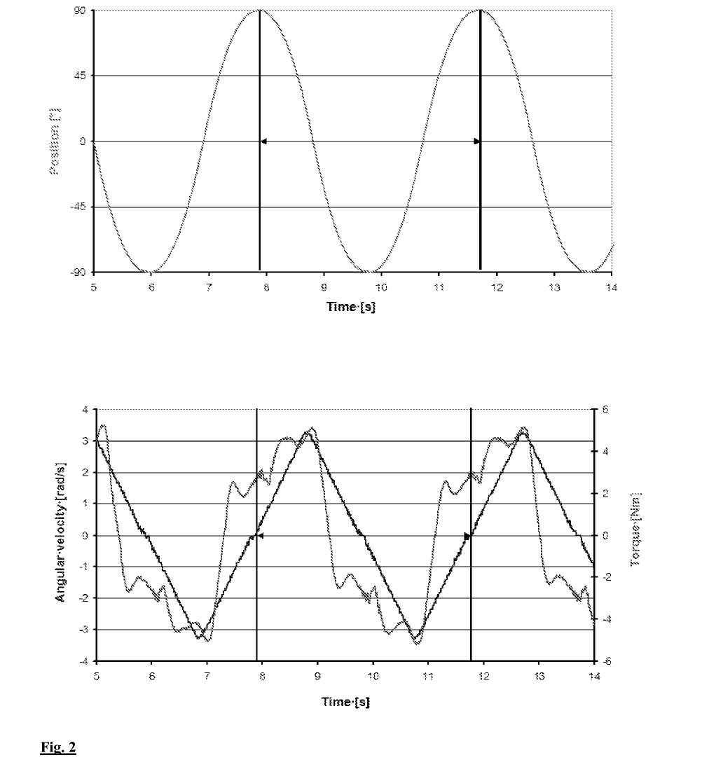

FIG. 2: Position, angular velocity and torque of a rotationally oscillating movement for light input, or optionally gasification and degassing of liquids.

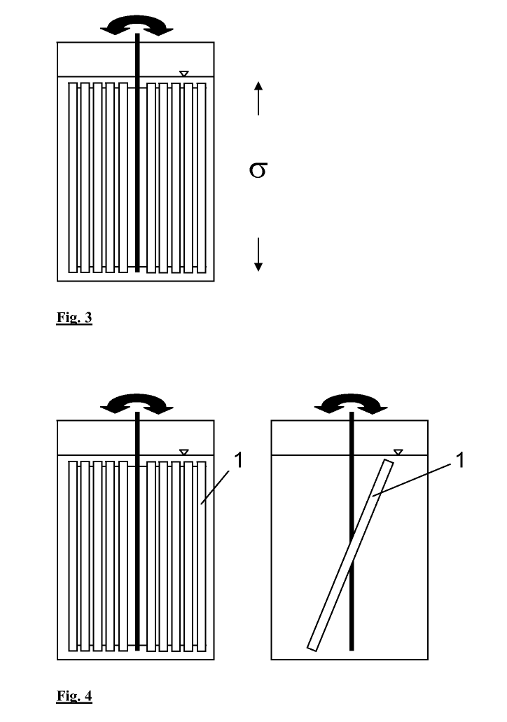

FIG. 3: Schematic representation of the device, characterized by a possibility of varying the tension σ of the light input surface, for example consisting of light guides and optionally of membrane tubes. Light guides wound on a rotor in this case form the light input surface.

FIG. 4: Schematic representation of the device, characterized by a possibility of varying the attitude angle of the light input surface. Light guides (1) wound on a rotor in this case form the light input surface.

FIG. 5: Schematic representation of the device, characterized by a possibility of limiting the excursion of the light input surface due to the flow resistance by control elements (4) in one rotation direction. Light guides (1) wound on a rotor in this case form the light input surface.

FIG. 6: Schematic representation of the device, characterized by a possibility of correspondingly shaping the light input surface for better mixing by control elements (4) and/or of applying stirring blades/paddles (5) or other devices for flow guidance and fixing. Light guides (1) wound on a rotor in this case form the light input surface.

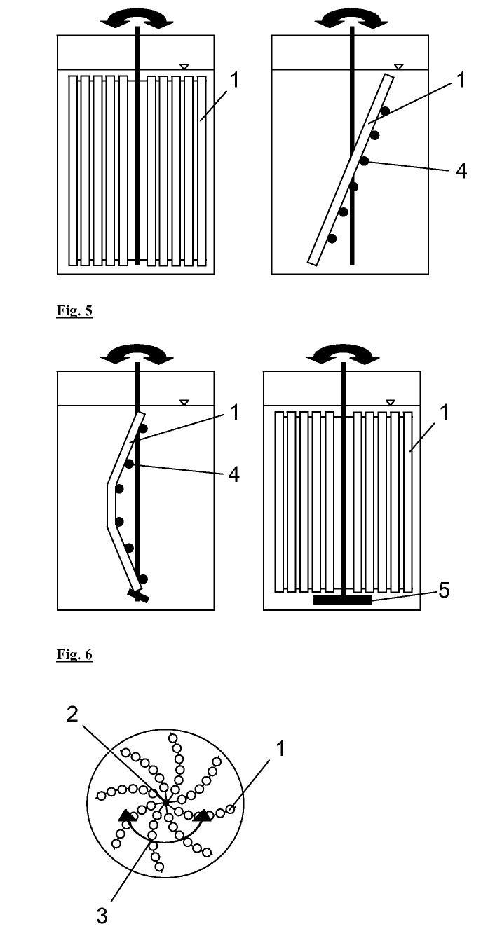

FIG. 7: Schematic representation of the device, characterized by a possibility of improving the mixing by configuring the rotor arms bent around the rotor shaft (2) in one of the rotation directions (3). Light guides (1) wound on a rotor in this case form the light input surface.

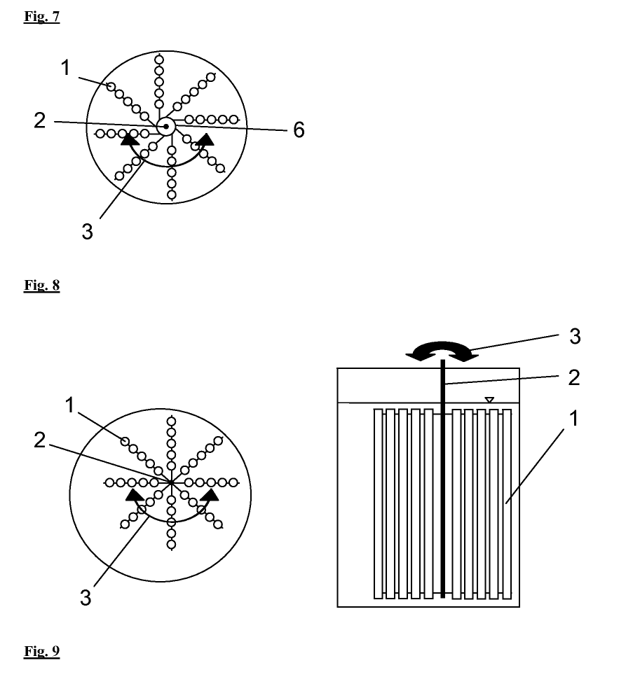

FIG. 8: Schematic representation of the device, characterized by a possibility of improving the mixing by applying the rotor arms tangentially around the rotor shaft (2) in one of the rotation directions (3) on a device (6). Light guides (1) wound on a rotor in this case form the light input surface.

FIG. 9: Schematic representation of the device, characterized by a possibility of improving the mixing by applying the rotor shaft (2) with the two rotation directions (3) off-centre in the container. Light guides (1) wound on a rotor in this case form the light input surface.

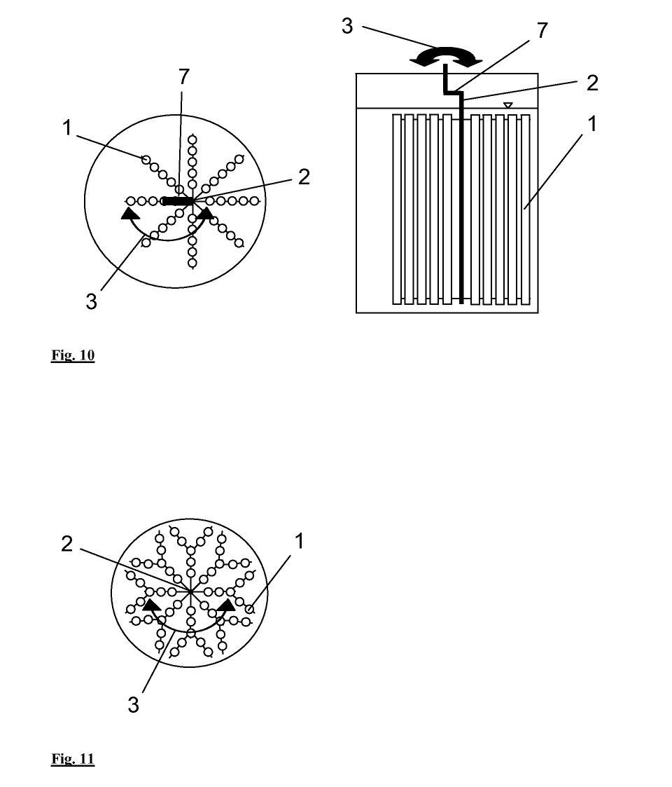

FIG. 10: Schematic representation of the device, characterized by a possibility of improving the mixing by the rotor shaft (2) with the two rotation directions (3) being applied centrally in the container but then having an eccentric (7). Light guides (1) wound on a rotor in this case form the light input surface.

FIG. 11: Schematic representation of the device, characterized by a possibility of distributing the light input area per unit volume as uniformly as possibly around the rotor shaft (2) with the two rotation directions (3). Light guides (1) wound on a rotor in this case form the light input surface.

References:
0—container of the photobioreactor
1—apparatus for light input, for example light guides, light sources, LEDs
1a—apparatus for gas transfer, for example membrane tubes
1b—apparatus for gas transfer, for example (micro)sparger
2—rotor shaft
3—rotation direction
4—control elements, by which the excursion of the light guides and optionally of the membrane tubes is limited in one rotation direction. These control elements may comprise light guides or LEDs.
5—stirrer
6—device for tangential arrangement of the rotor arms
7—eccentric in rotor shaft
σ—tension

The invention will be explained in more detail below with the aid of an exemplary embodiment, but without being limited thereto.

FIG. 1 schematically represents an example of a device for carrying out the method according to the invention. The light source is formed by light guides (1), which are arranged vertically on a rotor shaft (2) transversely to the rotation direction (3). By means of the flexible light guides, light from a light source, preferably arranged outside the bioreactor, can be guided into the interior where it emerges uniformly from the light guide. For example, gas containing carbon dioxide for supplying organisms such as algae and for transporting away oxygen which is formed may be conveyed through the optionally supplementary membrane tubes (1a in FIG. 1a and FIG. 1b). The membrane tubes and light guides may always be wound alternately and alternately with an offset on each neighbouring rotor arm (FIG. 1a) or, for example, light guides may be applied on every second rotor arm and membrane tubes on all the other rotor arms (FIG. 1b). A further possibility for gas transfer is to apply a sparger (1b) (for example a microsparger) below the light guide surfaces (FIG. 1c).

The light input device is preferably operated inside a photobioreactor (0) and the light generated is conveyed through continuous light guides into the photobioreactor, where the light emerges from the surface of the light guides wound on the rotor arms. The light guides are wound with a small spacing next to one another on the rotor arms. For orderly winding and to prevent slipping, the surface of the rotor arms is preferably provided with indentations.

Preferably, the light guides and optionally the membrane tubes are immersed fully in the culture medium. The device can execute a rotational movement about the rotor shaft (2). Preferably, it executes a rotationally oscillating movement. This movement leads on the one hand to improved supply of the organisms in the bioreactor with light and, with optional use of membrane tubes, to (bubble-free) gas transport, and on the other hand to greatly reduced susceptibility to the formation of deposits and agglomerates (compared with a static light surface and optionally membrane surface to which the flow takes place by means of a stirring mechanism).

FIG. 2 shows by way of example an oscillation of the rotor respectively through 180° in one direction and subsequently through 180° back into the starting position. This oscillation takes place with a constant angular velocity (FIG. 2). The resulting relative velocity between the rotor and the reactor content can be seen from the plotted torque (FIG. 2).

FIG. 3 shows a possibility of varying the tension σ of the light input surface, for example consisting of light guides and optionally of membrane tubes.

FIG. 4-6 show possibilities of varying the attitude angle (FIG. 4), limiting the excursion of the light input surface due to the flow resistance by control elements (4) in one rotation direction (FIG. 5) and correspondingly shaping the light input surface for better mixing by control elements and/or of applying stirring blades/paddles (5) or other devices for flow guidance and mixing (FIG. 6).

FIG. 7-10 show possibilities of improving the mixing - configuring rotor arms bent around the rotor shaft in one of the rotation directions (FIG. 7), applying rotor arms tangentially around the rotor shaft in one of the rotation directions on a device (6) (FIG. 8), applying the rotor shaft off-centre in the container (FIG. 9), applying the rotor shaft centrally in the container but with an eccentric (FIG. 10).

FIG. 11 shows a possibility of distributing the light input area per unit volume as much as possible in the container.

Preferably, 8 rotor arms are used, preferably with branching of the rotor arms as shown in FIG. 11.

1. A photobioreactor, comprising light sources arbitrarily mobile in rotational oscillation distributed in a bioreactor liquid volume.

2. The photobioreactor according to claim 1, wherein said light sources are spatially distributed approximately homogeneously.

3. The photobioreactor according to claim 1, wherein said light sources are formed by one or more light guides or light-emitting diodes in one or more membrane surfaces.

4. The photobioreactor according to claim 1, which comprises gas transport means mobile in rotational oscillation, selected from the group consisting of spargers, microspargers and membrane surfaces.

5. The photobioreactor according to claim 3, wherein said membrane surface is applied on rotor arms applied in a star shape on a rotor shaft.

6. The photobioreactor according to claim 3, comprising control elements by which the excursion of said membrane surface in one rotation direction can be limited.

7. The photobioreactor according to claim 5, wherein said control elements comprise light guides or light-emitting diodes.

8. The photobioreactor according to claim 1, wherein said light source, and/or a gas transport means are connected by means of a common frame to one or more probes.

9. A method of using the photobioreactor according to claim 1, comprising executing an arbitrary discontinuous movement using said light sources.

10. The method according to claim 9, wherein said light sources execute an arbitrary movement with movement reversal.

11. The method according to claim 9, wherein said light sources execute an arbitrary rotationally oscillating movement.

12. The method according to claim 9, wherein a gas transport means executes an arbitrary discontinuous movement, a movement with movement reversal or a rotationally oscillating movement.

13. The method according to claim 12, wherein said movements of the light sources, and/or the gas transport means comprise a periodic sequence of acceleration and deceleration between two movement turning points.

14. The method according to claim 9, capable of being used for the cultivation of algae or phototrophic organisms.

15. A method for promoting growth of phototrophic organisms in a photobioreactor comprising using light guides and/or light emitting diodes.
